(19) United States
(12) Patent Application Publication
(10) Pub. No.: US 2007/0176015 A1
(43) Pub. Date: Aug. 2, 2007

(54) System for delivering volatile materials

(75) Inventors: Michael Sean Farrell, Terrace Park, OH (US); Gerard Bernard Meyer, Cincinnati, OH (US); Hirotaka Uchiyama, Loveland, OH (US); Kim Lynn Webb, Fairfield, OH (US); Scott Edward Smith, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/654,996

(22) Filed: Jan. 18, 2007

Related U.S. Application Data
(60) Provisional application No. 60/763,449, filed on Jan. 30, 2006.
(60) Provisional application No. 60/765,428, filed on Feb. 2, 2006.

(51) Int. Cl. A24F 25/00; A61L 9/04; A62C 13/62

(52) U.S. Cl. 239/34; 239/302; 239/43

(57) ABSTRACT

A flippable/tiltable non-energised volatile material delivery system for delivery in a continuous manner and boost on demand, the system comprising:

A) a delivery engine comprising:
i) a liquid comprising at least one volatile material;
ii) at least two liquid reservoirs;
iii) a liquid flow retardant situated in the liquid flow path between the at least two liquid reservoirs and wherein the flow retardant has at least one evaporative surface; and
iv) a protective membrane adjacent to the retardant; and

B) optionally a housing encasing the delivery engine.

### CROSS REFERENCE TO RELATED APPLICATION

This application claims the benefit of U.S. Provisional Application No. 60/763,449, filed 30 Jan. 2006 and 60/765,428 dated 2 Feb. 2006.

### FIELD OF THE INVENTION

The present invention relates to a system for emitting or releasing volatile material to the atmosphere. More specifically, the invention relates to a system for delivering one or more volatile materials in a continuous manner and boost on demand using a non-energised delivery system.

### BACKGROUND OF THE INVENTION

It is generally known to use a device to evaporate a volatile composition into a space, particularly a domestic space, in order to deliver a variety of benefits, such as air freshening or perfuming of the air.

Non-energised systems, for example, systems that are not powered by electrical energy, are a popular way for the delivery of volatile materials into the atmosphere. The systems can be classified into: i) those that require human actuation, such as aerosols; and ii) those which do not required human actuation such as wick based systems and gels. The first type delivers the volatile materials on demand and the second type in a more continuous manner.

Methods and systems for releasing volatiles materials to the atmosphere, delivering a continuous maintenance level of emission and a temporary boost level of emission are known in the art. WO 05/032606 and WO 05/032607 disclose a delivery system for the delivery of a volatile material comprising two fluid reservoirs and a wick fluidly communicating the two reservoirs. A drawback with this kind of systems is that they are prone to leaks and have safety concerns associated with them. A second drawback is the lack of flexibility in the design of the system.

### SUMMARY OF THE INVENTION

According to a first aspect of the invention there is provided a delivery system for a volatile material. The system of the invention is suitable for delivering volatile material in a continuous manner and a boost and/or regeneration of volatile material on demand. The boost and/or regeneration of volatile material is/are achieved by flipping and/or tilting the system. The system delivers volatile material in a continuous manner when there is no human interaction, i.e., when the device is left to operate by itself. The system provides a boost and/or regeneration of volatile material by human interaction, i.e., by flipping and/or tilting it.

The system of the invention is preferably “flippable” and/or “tiltable”. By “flippable” and/or “tiltable” is meant that the system can be used in different spatial arrangements, which can be achieved by, for example, rotation (preferably inversion or inclination) of the system. The system is designed to be self-standing and fully operational at different inclinations and/or orientations, i.e., is a multi-positions system. A boost and/or regeneration of volatile material is/are provided by flipping or tilting the delivery system. By “boost” is understood an increase in the perceived intensity of volatile material. By “regeneration” is understood a change in the perceived character of the delivered material.

The system is “non-energised”. By non-energised is meant that the system does not require to be powered by a source of external energy. In particular, the system does not need to be powered by a source of heat, gas, or electrical current, and the volatile material is not delivered by aerosol means.

The delivery system comprises a delivery engine and optionally a housing encasing it. The delivery engine comprises:
i) a liquid comprising at least one volatile material;
ii) at least two liquid reservoirs, i.e., two different locations for storing liquid;
iii) a liquid flow retardant situated in the liquid flow path between the at least two liquid reservoirs and wherein the flow retardant has at least one evaporative surface; and
iv) a protective membrane adjacent to the retardant.

By “liquid reservoir” herein is meant a part of the delivery engine in which liquid can be stored. The at least two liquid reservoirs do not need to be two different receptacles, they can be part of the same receptacle that can store liquid in more than one location depending on the spatial orientation of the system. For example the liquid reservoir can be just one receptacle surrounding the flow retardant, having an oval, circular, square, rectangular, hexagonal shape or any irregular shape. The liquid can then be stored in different parts of the receptacle depending on its orientation.

The system of the invention provides great flexibility by allowing different modes of operation (continuous and on demand) with and without human interaction. Handling of liquid containing devices, especially when movement of the device (such as flipping or tilting) is involved, makes devices prone to leaks. Additionally, the devices of this type might encourage children to touch the delivery engine. The liquid composition might be unsafe, if a child put his hand into his mouth after touching it. The system of the invention, in particular the delivery engine, is leak resistant even if the delivery engine is pushed with a finger. Moreover, the system is free of leaks even when placed on a non-horizontal surface. The performance of the system is not altered by its position thereby providing flexibility of use.

The protective membrane not only makes the delivery engine leak free but also may provide some protection to the liquid components from oxygen which may degrade the liquid changing the character of the volatile material.

The delivery engine may be used as such or enclosed in a housing. This allows a great flexibility of design. Different configurations of the delivery engine can provide the benefits of the system of the invention. The outside housing can be designed independently of the delivery engine. The appearance of the system of the invention is very important, because it is usually used inside the home and in addition to the delivery of volatile materials the system can be considered as an ornamental and/or a fun piece. The delivery engine can be replaced or replenished while keeping the outside housing.

The flow retardant can be anything that increases the time that the liquid takes to travel from one liquid reservoir to another. The flow retardant comprises an evaporative surface from which volatile material is being constantly emitted.

The delivery system of the invention can be used for a variety of applications, including delivery of perfume, malodour elimination, pest control, aromatherapy, etc. In a preferred embodiment, the system is used for the delivery of perfumes. Perfume habituation is one of the problems faced when perfume is being delivered constantly, the human brain gets used to the perfume and cannot notice it anymore. This problem is overcome with the present device by providing a boost of volatile materials. Another problem found in systems for the delivery of volatile materials using evaporative surfaces is that different materials might evaporate at different times, if the evaporative surface is an absorbent medium, the medium can be clogged or blocked. These two problems are again avoided or ameliorated with the device of the invention by flipping and/or titling it.

In a preferred embodiment the liquid reservoirs are two independent receptacles and preferably the liquid reservoirs are placed one above the other. Preferably the ratio width/height of the liquid reservoirs is at least about 6:1, more preferably at least about 5:2. This provides a better control of the flow of the liquid into the flow retardant.

In a preferred embodiment each of the at least two liquid reservoirs are connected to the flow retardant by means of an aperture, preferably a channel or conduit. Preferably the conduit has a width considerably smaller than the width of the liquid reservoir. Preferably, the width of the reservoir is at least twice, more preferably at least five times and even more preferably at least eight times the width of the conduit. This contributes to slow down the entrance of the liquid into the flow retardant.

Although the flow retardant can have any configuration, a flat configuration is preferred from the surface area and efficiency of delivery points of view. By “flat” is meant that the width and height of the flow retardant are at least twice, preferably at least five times and more preferably at least eight times the thickness of the flow retardant.

In a preferred embodiment, the flow retardant is a wick. The wick may have any shape or configuration. Preferably, the wick is flat, for example having rectangular or square shape. It is also preferred the thickness of the wick being small compared with the remaining dimensions of the wick. Preferably the width of the wick is at least twice, more preferably at least five times and even more preferably at least eight time the thickness of the wick. It is also preferred that the wick does not extend into the liquid reservoirs. Systems comprising a wick can present the potential problem of providing distillative release of volatile materials from a composition, wherein the more volatile materials are released first, followed by materials of ever decreasing volatility. This leads to changes in the composition's character over the lifetime of the device. This problem can be reduced or even overcome with the system of the invention by flipping or titling the device, this help to the improve the homogeneity of the composition by mixing the materials by means of the liquid movement due to the flipping or tilting action.

In a preferred embodiment, the flow retardant is in contact with the membrane, this seems to favours the transfer of volatile material to the exterior.

Preferably, the protective membrane is vapour permeable and liquid impermeable thereby stopping wetting or leak problems.

The housing may be made of a material permeable to volatile materials or/and can have apertures, such as pin holes or small orifices for the delivery of the volatile materials.

In a method aspect of the invention, there is provided a method of delivering volatile material on demand by flipping and/or tilting the device of the invention.

### DETAILED DESCRIPTION OF THE INVENTION

The present invention envisages a system which delivers volatile material in a continuous manner while transferring liquid comprising at least one volatile material from one liquid reservoir to another and additionally permits to provide a boost and/or regeneration of the volatile material by flipping and/or tilting the device.

The system of the invention is suitable for purposes of providing fragrances, air fresheners, deodorizers, odor eliminators, malodor counteractants, insecticides, insect repellants, medicinal substances, disinfectants, sanitizers, mood enhancers, and aromatherapy aids, or for any other purpose using a material that acts to condition, modify, or otherwise change the atmosphere or the environment. The at least one volatile material provided by the system of the invention may be from a single source, or alternatively from multiple sources. The one or more volatile materials may have various volatility rates over the useful life of the delivery system.

The term “volatile material” as used herein, refers to a material that is vaporizable at room temperature and atmospheric pressure without the need of an energy source. Any suitable volatile material in any amount or form may be used. Liquids suitable for use herein includes (but are not limited to) compositions that are comprised entirely of a single volatile material or compositions that have more than one volatile component, and it is not necessary for all of the component materials of the liquid to be volatile. Liquid suitable for use herein may, thus, also have non-volatile components, such as carrier materials (e.g., water, solvents, etc). It should also be understood that when the liquid is described herein as being “emitted” or “released,” this refers to the volatilization of the volatile component thereof, and does not require that the non-volatile components thereof be emitted.

The volatile material can be in the form of perfume oil. Most conventional fragrance materials are volatile essential oils. The volatile material can be a volatile organic compound commonly available from perfumery suppliers. Furthermore, the volatile material can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, lavandin, neroili, rose absolute, and the like. In the case of air freshener or fragrances, the different volatile materials can be similar, related, complementary, or contrasting.

The volatile material may also originate in the form of a crystalline solid, which has the ability to sublime into the vapor phase at ambient temperatures or be used to fragrance a liquid. Any suitable crystalline solid in any suitable amount or form may be used. For example, suitable crystalline solids include but are not limited to: vanillin, ethyl vanillin, coumarin, tonalid, calone, heliotropene, musk xylol, cedrol, musk ketone benzohenone, raspberry ketone, methyl naphthyl ketone beta, phenyl ethyl salicylate, veltol, maltol, maple lactone, proeugenol acetate, evemyl, and the like.

It may not be desirable, however, for volatile materials to be too similar if different volatile materials are being used in an attempt to avoid the problem of emission habituation, otherwise, the people experiencing the emissions may not notice that a different emission is being emitted. The different emissions can be related to each other by a common theme, or in some other manner. An example of emissions that are different, but complementary might be a cinnamon emission and an apple emission. For example, the different emissions can be provided using a plurality of delivery systems each providing a different volatile material (such as, musk, floral, fruit emissions, etc).

The delivery system is preferably sealed precluding the transfer to the exterior of liquid and only allowing the transfer of volatile material.

The delivery system of the invention emits volatile materials in a substantially continuous manner when the system is in a resting position, i.e., the system is not being flipped, tilted, shaken or otherwise moved. The emission level of volatile material may exhibit a uniform intensity until substantially all the volatile materials are exhausted from the liquid at substantially the same time. This uniformity can be altered by flipping and/or tilting the device, thereby altering the delivery profile, i.e., the intensity and/or character of the emission. The delivery of the maintenance level emission can be of any suitable length, preferably at least 24 hours, more preferably at least 2 days and most preferably at least 1 week.

When the boost level emission mode is activated by human interaction, i.e., by flipping and/or tilting the system, a higher, optionally uniform, intensity of volatile material is emitted over a suitable emission duration, at which time the delivery system can automatically return to delivering volatile material in the maintenance level emission mode without further human interaction. The term “temporary” with regard to the boost level emission, means that though it is desirable for the boost level emissions to emit at a higher intensity for a limited period of time after being activated and/or controlled by human interaction, the boost level emission can also include periods where there are gaps in emissions. Not to be bound by theory, it is believed that the higher intensity of the boost level emission depends upon a number of factors. Some of these factors include, but are not limited to: the “perfume effect” of the volatile material; the volume of the volatile material delivered to the evaporative surface device for purposes of providing a boost level emission; the rate of delivery of the volatile material available from the source for boost level emissions; and the available surface area of the evaporative surface device during the delivery of the boost level emission.

Any suitable volatile material, as well as, any suitable volatile material volume, rate of delivery, and/or evaporative surface area may also be used to raise and/or control the intensity of the boost level emission. Suitable volumes, rates of delivery, and surface areas are those in which the boost level emission exhibits an emission intensity greater than or equal to the maintenance level emission. For example, by providing a greater volume of volatile material to the evaporative surface device, the intensity of the boost level emission may be an increased and/or controlled by the consumer. The volume of the volatile material delivered to the evaporative surface device may also be controlled using a specific dosing device having a specific volume. A collection basin may be used to force a certain volume through the evaporative surface device. The collection basin may be made of any suitable material, size, shape or configuration and may collect any suitable volume of volatile material. For example, the delivery system may comprise a collection basin, such as a unit dose chamber, that may be at least partially filled with at least some of the volatile material to activate the boost level emission. The unit dose chamber provides a controlled volume of the volatile material to an evaporative surface device, such as an evaporative surface device.

Liquid flow retardants suitable for use herein include any suitable means that delays the flow of the liquid from one liquid reservoir to the other, including channels of different diameters, structures providing a liquid tortuous path, liquid stoppers and surface devices having any suitable size, shape, form, or configuration. The liquid flow retardants include at least one surface that allows for at least some evaporation of volatile material. The flow retardant may be a separate element from the liquid reservoir or may be part of a liquid reservoir. Suitable flow retardants made from any suitable material, include but are not limited to: natural materials, man-made materials, fibrous materials, non-fibrous materials, porous materials, non-porous materials, and combinations thereof. Preferred flow retardants for use herein include absorbent materials, in particular wicks capable of being impregnated with the liquid containing volatile material. The liquid is absorbed into the absorbent material by capillary action. Examples of absorbent material are blotter paper, felt, cellulose, cotton, wood chips, dried vegetation, sponge material, polymer, copolymer and other porous or fibrous material. Preferred for use herein are water-insoluble polymers such as low-density polyethylene, high-density polyethylene, copolymers of ethylene acetate and vinyl acetate, polypropylene, polyvinyl chloride, cellulose acetate, methyl cellulose, cellulose acetatebutyrate polyisobutylene, etc. The absorbent can be shaped or presented in an attractive or decorative manner. The absorbent material can be hollow. This arrangement, allows excess liquid to flow through the center. The absorbent material can have areas of different densities. This can help with flow control.

In some non-limiting embodiments, the wick is an aligned fibers wick (i.e. “sintered”). Such wicks allow improved perfume containment (vs. a compression or “amorphous” wick), by increasing the directionality of the volatile material flow. In one embodiment, the aligned fibers wick is made from a polyester/polyolefin blend. Such blends absorb less of the perfume composition's components than pure polyester, thus providing faster movement of any perfume excess and reducing the incident of wick saturation and leakage. In another embodiment, the wick has an average density of from about 1 g/cm3 to about 0.01 g/cm3. Preferably, the wick has an average density of less than about 0.5 g/cm3, more preferably less than about 0.02 g/cm3. Wicks having a high density, i.e, more than 0.5 g/cm3 can contribute to flow control.

Preferably, the delivery system maintains its character fidelity over time with periodic reversals in volatile material flow direction on the evaporative surface device. For example, over time the character fidelity of the delivery system may decrease due to fractionation (such as, partitioning effects) of at least one volatile material or by clogging or blocking of the evaporative surface. The solution to both fractionation and clogging or blocking is to provide a suitable flow direction change or reversal on the evaporative surface device over a suitable duration by flipping and/or tilting the device. For example, a suitable flow reversal of the evaporative surface device may consist of the activation of the boost level emission and emission over a suitable duration. In this case, volatile material flow reversal of the evaporative surface device resulting from flipping, tilting or any other mechanism can substantially flush the evaporative surface in a manner sufficient to clear away some of the unwanted insoluble precipitates, fractionation and/or partitioning effects. Thus, character fidelity is at least partially restored by flushing the evaporative surface during the boost level emission. In this way, the consumer can revive the dynamic interactive scent experience by sensing the entire range of different volatile materials contained in the delivery system in a simple step.

The housing preferably permits visual inspection of the delivery engine. This is preferred for aesthetic reasons and provides an element of fun. The housing can be made of any suitable material such as glass, ceramic, wood, plastic, composite material, etc, and can have any size, shape and configuration suitable for encasing the delivery engine. The housing can be rigid or flexible and can be made of material which allows the transfer of volatile materials to the surrounding environment. The housing preferably has apertures which help to control the delivery of volatile material. Increasing the effective size of the housing apertures, will increase the delivery of volatile material. Conversely, decreasing the effective size of the apertures, will decrease the delivery of volatile material. Preferably, the number and/or size of the apertures is not fixed but can be controlled by the user, by means of for example sliding doors. Preferably, the shape of the housing is such that can stand on one of its basis before and after it has been flipped. The housing preferably has two or more self-standing positions.

The protective membrane may be made of any permeable polymeric, thermoplastic, or thermoset material, including acetal, acrylic, cellulosic, fluoroplastic, polyamide, polyester, polyvinyl, polyolefin, styrenic, etc, alone, co-extruded, woven or non-woven, mixed or in combination with elastomers, rubber, solids, silicone or combinations thereof.

Preferably the protective membrane for use herein is permeable to volatile material and impermeable to liquid, more preferably, the membrane is a composite microporous membrane a of particular construction allowing it safely to contain volatile dispersant emitting substances in solid or liquid form while permitting the uniform dissipation of gaseous or suspended dispersant to the surrounding environment. The membrane is placed adjacent to the retardant, preferably the membrane is parallel to the retardant. A headspace can exist between the membrane and the retardant. For performance reasons, it is preferred to have the membrane and retardant in close proximity, more preferably in contact with one another.

Preferred membranes for use herein comprise a backing material, such as polymeric nonwoven, a permeable membrane such as expanded polytetrafluoroethylene film, and a coating such as a polytetrafluoroethylene resin. When combined in various forms in the manner disclosed in U.S. Pat. No. 5,497,942, these elements produce a composite which resists wetting by low surface tension liquids and provides a regular and uniform surface for the release of a wide variety of dispersant materials.

A preferred permeable membrane comprises a porous or permeable polymeric layer, for example a polyolefin and particularly a fluoropolymer such as polytetrafluoroethylene (PTFE), co-polymers of PTFE and/or other fluoropolymers, perfluorodioxole polymer, etc. Ideally the membrane comprises a membrane of PTFE which has been expanded to form a permeable network of polymeric nodes and fibrils. This material can be made in any known manner, such as in the manner disclosed in U.S. Pat. No. 3,953,566. As is known, this material has the unique ability of being water proof and moisture vapour permeable. As such, it has been determined that volatile material will freely dissipate through this membrane while the liquid will be safely contained within the delivery engine.

Expanded PTFE material is commercially available from W. L. Gore & Associates, Inc., Elkton, Md., in a variety of forms under the trademark GORE-TEX or ZINTEX.

Other suitable membrane is the microporous polymeric membrane described in FR 2,754,455 having a porous diameter of from about 0.1 to about 5 μm and that has been subjected to a treatment to render it super-hydropobic and oleophobic.

In preferred embodiments the delivery system comprises two liquid reservoirs, one above the other. The top reservoir has an aperture, preferably a channel, at the bottom and the bottom reservoir has an aperture, preferably a channel at the top. A flow retardant preferably in the form of an absorbent surface such as a wick, preferably a flat wick, is placed between the two apertures. Preferably the liquid reservoirs are not flushed with the wick but have an additional back space, contributing to flow control. When the delivery engine is in its upright position the liquid flows through the top aperture into the absorbent surface wetting it. The volatile material evaporates from the absorbent surface. A permeable membrane, preferably vapour permeable and liquid impermeable, is placed between the absorbent surface and the exterior. The volatile material crosses the membrane and gets delivered into the surrounding atmosphere. The remaining liquid is collected in the bottom reservoir. The process can be repeated by rotating the engine 180°. Preferably, the delivery engine is encased by a housing. It is also preferred a housing having front, back, top, bottom and sides, for example a parallelepiped, preferably having a quasi rectangular or square two-dimensional profile. Preferably, the housing has orifices, preferably of a diameter smaller than about 8 mm, on the front and back. It is also preferred that the housing has top, bottom and/or sides apertures to favour the air flow through the device improving the delivery of the volatile material.

The system may also comprise more than two reservoirs which can be filled with the same or different compositions. The reservoirs may have any configuration, they can be at 45°, preferably 90° and more preferably 180° with respect to one another.

The delivery engine is sealed to liquid and permits the transfer of volatile material to the exterior through the protective membrane. The body of the delivery engine can be made of any material, plastic and in particular coloured or non-coloured see-through plastic is preferred for use herein. The see-through material permits observation of the liquid flowing from one reservoir to the other.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A flippable/tiltable non-energised volatile material delivery system for delivery in a continuous manner and boost on demand, the system comprising:
A) a delivery engine comprising:
i) a liquid comprising at least one volatile material;
ii) at least two liquid reservoirs containing said liquid and having said liquid flow path therebetween;
iii) a liquid flow retardant situated in the liquid flow path between the at least two liquid reservoirs and wherein the flow retardant has at least one evaporative surface; and
B) a housing encasing the delivery engine.

2. A system according to claim 1 wherein the liquid is a perfume composition.

3. A system according to claim 1 wherein the at least two liquid reservoirs are two independent receptacles.

4. A system according to claim 1 wherein the at least two liquid reservoirs are situated one above the other and are connected to the flow retardant by means of an aperture.

5. A system according to claim 1 wherein the flow retardant has a flat configuration.

6. A system according to claim 1 wherein the flow retardant is a wick.

7. A system according to claim 1 wherein the flow retardant is in contact with the membrane.

8. A system according to claim 1 wherein the membrane is vapour permeable and liquid impermeable.

9. A system according to claim 1 wherein the housing comprises apertures for the delivery of the volatile material.

10. A method of delivering a boost of volatile material on demand using the device of claim 1 comprising the step of flipping and/or tilting the device.
